(19) United States
(12) Patent Application Publication
(10) Pub. No.: US 2003/0224455 A1
(43) Pub. Date: Dec. 4, 2003

(54) Method for the diagnosis of heart diseases

(76) Inventors: Mariapia Abbracchio, Milano (IT); Flaminio Nicola Cattabeni, Milano (IT); Pier Andrea Borea, Ferrara (IT); Katia Varani, Pontelagoscuro (IT); Franco Laghi Pasini, Siena (IT); Alessandra Camurri, Milano (IT)

Correspondence Address: ABELMAN, FRAYNE & SCHWAB, 150 East 42nd Street, New York, NY, 10017-5612, US

(21) Appl. No.: 10/156,352

(22) Filed: May 28, 2002

(51) Int. Cl. G01N 33/53; G01N 33/567

(52) U.S. Cl. 435/7.2

(57) ABSTRACT

The present application discloses a method to diagnose heart diseases, characterised by measuring, in the affected cells, the density of A_{2A} receptors and/or the production of cyclic AMP after stimulation of these cells with A_{2A} receptor agonists. According to the invention, these parameters are used as markers for monitoring the onset, progression and remission of heart diseases. The circulating cells in the patients blood were found to be an adequate model for monitoring, at a peripheral level, the course of these pathologies: this allows the assay to be performed in the way of a simple blood test. The method allows to detect the aforesaid diseases even in their earliest stages, thus allowing the possibility of a timely treatment; the efficacy of any chosen treatments can also be monitored by the present method.

### FIELD OF THE INVENTION

The invention disclosed herein belongs to the field of diagnosis and therapy of cardiovascular disorders. In particular, this invention relates to a method for the diagnosis of heart diseases involving a haemodynamic deficit. Examples of such diseases are terminal heart failure, acute myocardial infarction, or cardiac hypofunctionality subsequent to heart transplantation.

### STATE OF THE ART

Both the adenosinergic system and certain inflammatory cytokines play an important role in the heart failure pathophysiology In the left ventricular insufficiency, the concentration of inflammatory cytokines in the blood increases with increasing severity of disease. It has been proved that elevated serum tumor necrosis factor-alpha (TNF-alfa) and interleukin-6 (IL-6) concentrations are predictive of subsequent development of complications in patients with heart failure (Deswal A., et al., Circulation, 103, 2055-2059, 2001; Rauchhaus M., et al., Circulation, 102, 3060-3067, 2000). Experimental studies demonstrated that such cytokines exert direct toxic effects on the myocardium. Transgenic mice overexpressing the gene for TNFalfa develop dilated cardiomiopathies with increase in myocardial apoptosis. Similarly, an overproduction of pro-inflammatory cytokines has been observed in the course of myocardial ischemia, and particularly in patients with complicated intrahospital course. It is not known whether the serum determination of such markers possesses an independent predictive value. The cardioprotective effects of the adenosine have been recognized since several years. Among the various cardiovascular protective mechanisms, it is well known that activation of the A_{2A} adenosine receptor reduces the production and release of proinflammatory cytokines in the blood from macrophages, lymphomonocytic and myocardial cells. In particular, adenosine influences the production of TNFalfa and IL6 by macrophages stimulated with LPS, reduces the expression of TNFalfa in failing heart, as well as that induced by myocardial infarction, and prevents TNFalfa-dependent myocardial dysfunction (Meldrum D. R., et al., J. Immunol., 92, 472-477,1997).

Preliminary results on the administration of adenosine have confirmed that this nucleoside plays a potential role also in human cardioprotection (Meldrum D. R., et al., American J. Physiol., 274, 577-595,1998.).

While a large body of work is available on the etiology of the diseases and on the search for suitable drugs, minor attention has been dedicated to biochemical tests capable of detecting the onset of these diseases, especially at their early stages, i.e. at subclinical level; this would be most interesting in order to provide patients with an early and thus more effective treatment; this need is particularly felt in the case of some heart diseases like terminal heart failure, for which no effective treatment is practicable once the disease is in full course.

### SUMMARY OF THE INVENTION

This invention is based on the identification of an anomalous behaviour of the A_{2A} adenosine receptor and its transduction system (adenylate cyclase enzyme) in patients with heart diseases characterised by an impaired haemodynamic function. The anomalous behaviour becomes experimentally evident as an increased density of A_{2A} receptors in the affected cells, and an overproduction of cyclic AMP after stimulation of these cells with an A_{2A} receptor agonist. The present invention uses these parameters as markers for the monitoring of the onset, progression and remission of the disease after suitable treatment. In particular, the cells circulating in the peripheral blood proved to be an adequate substrate for monitoring the pathologic events taking place in the heart. The present invention allows, via a simple blood test, to detect the aforesaid heart diseases even in their earliest stages, thus allowing the possibility of a timely treatment and of monitoring the efficacy of therapeutic interventions.

### DESCRIPTION OF THE FIGURES

FIG. 1. Density (A) and affinity (B) of A_{2A} adenosine receptors in lymphocytes taken from control healthy subjects and subjects (4) with terminal heart failure (CHF) analysed both prior to and subsequent of heart transplantation.

FIG. 2. Increase in the A_{2A} receptor expression in peripheral blood mononuclear cells in patients with heart failure.

### DETAILED DESCRIPTION OF THE INVENTION

Object of the present invention is an easy-to-perform and sensible assay for monitoring the development of heart diseases involving a haemodynamic deficit. The assay allows to promptly diagnose a pathological condition, even when at a subclinical level, thus enabling an early pharmacological treatment; the efficacy of any chosen treatment can also be monitored by the present assay throughout the entire treatment until complete restoration of the haemodynamic function. The invention is based on the finding, made by the Applicant, that the onset and development of heart diseases characterised by a haemodynamic deficit is signalled by specific biological events taking place both in heart tissue cells and in the peripheral blood cells of the patient. These events are: (a) an increase in density of A_{2A} receptors and (b) an overproduction of cyclic AMP induced by A_{2A} agonist compounds; both these events are connected to an increased activity of the A_{2A} receptor, occurring in the patients suffering from the aforesaid diseases. It is thus object of the present invention a method to monitor in patients the onset, development and regression of a heart disease involving haemodynamic deficit, this method being characterised by measuring, in the affected cells, the density of A_{2A} receptors and/or the production of cyclic AMP induced by A_{2A} agonist. An increase of these parameters with respect to basal levels (i.e. those of healthy control cells) is an indication of a pathological status; the extent of increase from the basal levels is an indication of the seriousness of the disease; a recovery of the basal levels after this increase is an indication of regression of the disease, such as can be the case after proper pharmacological treatment or heart transplantation. Therefore, both the above parameters (a) and (b) are used as biological markers for monitoring the disease progression on the one side, and the restoration of a normal haemodynamic function on the other.

For the purpose of the present invention, the measurement of one of the two aforesaid parameters provides sufficient diagnostic information on the disease; however, if desired, both parameters can be measured, so to provide a double-checked result: this is particularly useful when the deviation from basal levels is most difficult to detect, i.e. when the disease is at its earliest stage or is almost completely healed.

Any heart disease involving, as a cause or consequence, a haemodynamic deficit, i.e. a lowered blood supply to tissues and organs, can be diagnosed with the present method. In particular, the conditions of cardiac hypofunctionality (especially the one occurring after heart transplantation, with or without rejection), myocardial infarction, and terminal heart failure are most effectively detected by the present method.

An important advantage of the present invention is that the aforementioned biological events (a) and (b) not only were found to take place in the heart cells, but also in the circulating cells of the peripheral blood of the diseased patients, in particular lymphocytes and neutrophils; thus in the most advantageous embodiment of the invention, the measure of the A_{2A} receptor density/cAMP production is performed on circulating blood cells of the patient, i.e. via a simple blood test.

In this case the method of the invention comprises the steps of: withdrawing a blood sample from the patient, separating from the plasma the particulate fraction, in particular lymphocytes and/or neutrophils, and measuring on this fraction the density of A_{2A} receptors and/or the production of cyclic AMP induced by A_{2A} agonists.

The isolation of the particulate fraction of the blood is generally performed within 6-8 hrs after withdrawal if a fresh blood sample is used, or at any time in case of frozen blood samples. The blood samples are stabilised and repeatedly centrifuged/resuspended in suitable buffers, as well known in the art, to obtain the desired cell fraction. In order to measure the density of A_{2A} receptors, the isolated cell fraction, preferably the one containing lymphocytes and/or neutrophils, can be further high-speed centrifuged to form a membrane on which the A_{2A} receptor density is conveniently measured. The receptor density (B_{MAX}) is thus calculated by known means, e.g. receptor binding techniques (Kenakin T. (1997) Drug receptor theory 1-38 In “Pharmacologic analysis of drug-receptor interaction”, Raven Press, New York). In alternative or in addition to this method, the receptor density can also be monitored by other techniques, e.g. via semi-quantitative RT-PCR, whereby an increased receptor density is signalled by an enhancement of the mRNA encoding for the receptor protein A_{2A}. Some examples of these determinations are shown in the experimental part below.

In order to measure the production of cyclic AMP a suitable A_{2A} agonist the compound N-ethylcarboxamidoadenoside (NECA) is incubated for 10 min at 37° C. in the presence of adenosine deaminase. At the end of the incubation, the cAMP levels can be measured by methods known in the art, e.g. as described in Varani K. et al., Br. J. Pharmacol., 122, 386-392, 1997. The degree of activity of the adenylate cyclase system is conveniently measured in terms of EC₅₀ for the chosen agonist: the lower the EC₅₀, the higher the pathologic status. Some examples of these determinations are shown in the experimental part below.

The method herein disclosed is especially useful to diagnose the occurrence of heart failure and other heart diseases well before the establishment of their life-threatening effects, thus allowing a more effective preventive and/or curative treatment; if the disease is in full course, the method is useful to assess the degree of haemodynamic deficit of the patient, and to highlight any changes (improvement/worsening) in this condition; in the case of heart-transplanted patients, the method allows to follow the normal course of recovery of haemodynamic functions and, if necessary, to provide the patient with adequate and timely treatment. The easiness in obtaining cell samples from the peripheral blood, and the simplicity in the measurement of the parameters of A_{2A} receptor activity, makes of this method a convenient tool to diagnose heart diseases characterized by haemodynamic deficit: for example, a sequence of tests can easily be performed with no discomfort for the patient, yielding a time curve showing the progression/healing of the disease over a given period of time; this provides the physician with an enhanced diagnostic response (dynamic picture of the disease) which is much more informative than the common diagnosis based on checking the mere presence/absence of symptoms.

While the withdrawal of peripheral blood is the most practical method to obtain the cells to be tested in the method of the present invention, the latter is not to be limited to this sampling procedure; the cells to be tested can also be obtained directly from the heart, if necessary; in this regard, it should be noted that the present method is not limited to the diagnosis of heart diseases in living patients, but can also be applied e.g. to hearts stored for transplantation, in order to assess their functionality, or to hearts of dead persons for establishing the cause of death. A further object of the present invention is a kit to perform the above described method. The kit contains instruction on the performance of the above described method, and further contains devices and substances necessary to perform it, such as a sterile syringe with needles to withdraw the blood, blood stablilizers, anticoagulants, centrifugation vials, suitable buffers to suspend cellular fractions, incubation medium for the cells, a unit dose of radiolabelled ligand useful for the determination of A_{2A} receptor density, a unit dose of A_{2A} agonist compound useful for the determination of the production of cAMP, etc.

The above presented invention is further disclosed and commented upon on the basis of the results shown in the experimental section below.

### EXPERIMENTAL PART

### Material and Methods

1. Isolation of the Circulating Cells of the Peripheral Blood

The isolation of the cellular fractions is started not later than 6-8 hours after the blood is collected from the patients (or the control healthy subjects compatible for age and sex). After stabilization with 1.4% citric acid, 2.5% citrate sodium and 2% glucose, the blood is centrifuged at 200 g for 10 minutes to obtain a platelet-rich plasma (PRP). The lymphocytes are separated from the monocytes and neutrophils by stratifying the blood using Ficoll-Hypaque gradient. Numerous centrifugations and resuspensions in phosphate buffer saline (PBS) are carried out in order to obtain the purified lymphocyte fraction. The pellet resulting from the previous resuspension and containing the erythrocytes is duly mixed with Dextran T500 and kept at room temperature for 60 minutes to make the erythrocytes precipitate to the bottom. The surface layer containing the neutrophils is removed and centrifuged several times to obtain a neutrophil-enriched cell suspension. Finally, the lymphocytes and neutrophils are partially destined to the preparation of membranes and undergo a series of homogenisations and centrifugations at high speed. The presence of A_{2A} adenosine receptors is determined in this membrane suspension according to the receptor binding technique. The cells (lymphocytes and neutrophils), duly dissolved (10⁶ cells/tube), are used in the experiments for the measurement of cAMP levels, where the function of the A_{2A} adenosine receptors is determined (Varani K. et al., Br. J. Pharmacol., 117, 1693-1701, 1996; Varani et al, 1997 op. cit.; Varani K., et al., Br. J. Pharmacol., 123, 1723-1731, 1998).

2. Measurement of the A_{2A} Recetor Binding

The saturation experiments are carried out incubating the isolated cells with 8-10 various concentrations of the [³H]-ZM 241385 antagonist in the concentration range between 0.01 and 10 nM. The non-specific binding is determined in presence of ZM 241385-1 μM. The samples are incubated for 60 minutes at 4° C. The free ligand is separated from the bound ligand by rapid vacuum filtration onto glass-fibre Whatman GF/B filters using a Brandel Harvester. The radioactivity on the filters is determined using a scintillation counter (Beckman 55).

3. Measurement of cAMP Levels

The cells are re-suspended in PBS buffer containing 2 U.I. of adenosine deaminase and pre-incubated for 10 minutes at 37° C. Then, increasing concentrations of N-ethyl-carboxamide adenosine (NECA, 1 nM-10 μM) or other adenosine analog are added. After 10 min a 6% trichloroacetic acid solution (TCA) is added to stop the reaction. The TCA suspension is centrifuged at 2000 g for 10 minutes at 4° C. and the supernatant is transferred into special extraction tubes with water-saturated ether. The final water solution is tested for measurement of cAMP levels according to the method disclosed in Varani et al., 1997, op. cit. The tubes in which the test is carried out contain: 100 μI sample, 125 μl of buffer consisting of 100 μM trizma base, 6 mM 2-mercaptoethanol, 8 mM aminophylline, pH 7.4, 25 μl [³H]-cAMP (corresponding to approx 20.000 cpm) and 100 μl cAMP binding protein, prepared using bovine suprarenal capsules. Then, a calibration curve is performed comprising: a) known quantities of non radioactive cAMP; b) a blank that does not contain the binding protein; c) the standards containing 0, 1, 2, 4, 7, 10 pmoles of non radioactive cAMP respectively. After stirring, the samples are incubated for 90 minutes at 4° C. The competitive binding of radioactive cAMP and non-radioactive cAMP to the protein is stopped by adding 100 μl activated carbon suspension at 10% in buffer containing 2% bovine albumin. The samples are centrifuged at 2.000 rpm for 10 minutes, then 200 μI supernatant is transferred in vials with scintillation liquid (Ready gel, Beckman). The corresponding cAMP concentration is determined by comparison with the calibration curve.

4. Semiquantitative RT-PCR Assessment of A_{2A} Receptor Expression

After the separation of the mononuclear cells from the blood by centrifugation on Lymphoprep gradient (Di Renzo M. et al. European Neurology. 45(3):192-3, 2001.), RNA is extracted using Triazol (GIBCO). The extracted RNA is determined by spectrophotometer reading at 260 nm; 250 ng RNA is reverse transcripted into cDNA using the enzyme M-MLV Reverse Transcriptase (GIBCO); the mRNA specific for the A_{2A} receptor is subsequently PCR-amplified using specific primers for the human A_{2A} receptor: (A2AFW: 5′-TGTCCTGGTCCTCACGCAGAG-3′; A2ARev: 5′-CGGATCCTGTAGGCGTAGATGMGG-3′). An optimal amplification protocol has been set up (it consists of 34-38 cycles of: denaturation at 95° for 60 seconds, annealing at 55° for 60 seconds and extension at 72° for 60 seconds) in presence of 32PdCTP, which is added to the reagents during the amplification cycles to obtain radiolabelled products. The amplified products are then separated using 6% polyacrylamide gel electrophoresis in Tris-borate-EDTA (TBE), and after being dried, the gels are exposed to autoradiographic films. The analysis of the autoradiographic films is carried out by means of an image-analysis software (QuantityONE, Byorad) capable of detecting incorporation of radioactivity in a band of 630 base pairs having the molecular weight as the expected amplification product.

### Results

Table 1 below shows the binding parameters of [³H]-ZM 241385 radioligand to A_{2A} receptor in membranes of lymphocytes and neutrophils taken from control subjects and patients with terminal heart failure (CHF).

TABLE 1

| | Lymphocyte membranes | | Neutrophil membranes | |
|---|---|---|---|---|
| | | B_{MAX} | | B_{MAX} |
| | | (fmol/mg | | (fmol/mg |
| Subjects | K_{D}(nM) | protein) | K_{D}(nM) | protein) |
| CONTROLS | 0.86 ± 0.03 | 48 ± 2 | 0.95 ± 0.02 | 54 ± 4 |
| n = 20 | | | | |
| CHF | 2.35 ± 0.10* | 82 ± 4* | 2.25 ± 0.10* | 85 ± 5* |
| n = 20 | | | | |

*P < 0.01, Student's t test

Table 2 below shows the stimulation of cAMP levels by NECA in lymphocytes and neutrophils taken from control subjects and patients with terminal heart failure (CHF).

TABLE 2

| | Lymphocytes | Neutrophils |
|---|---|---|
| | EC₅₀ | EC₅₀ |
| Subjects | (nM) | (nM) |
| CONTROLS | 245 ± 10 | 250 ± 10 |
| n = 10 | | |
| CHF | 134 ± 10* | 120 ± 10* |
| n = 10 | | |

*P < 0.01, Student's t test

Table 3 below shows the binding parameters of [³H]-ZM 241385 radioligand to A_{2A} receptor in heart membranes taken post-mortem from control subjects and from transplanted patients with terminal heart failure (CHF).

TABLE 3

| Subjects | K_{D} (nM) | Bmax (fmol/mg protein) |
|---|---|---|
| CONTROLS | 2.35 ± 0.10 | 130 ± 10 |
| (n = 8) | | |
| CHF | 4.20 ± 0.20* | 210 ± 10* |
| (n = 8) | | |

*P < 0.01, Student's t test

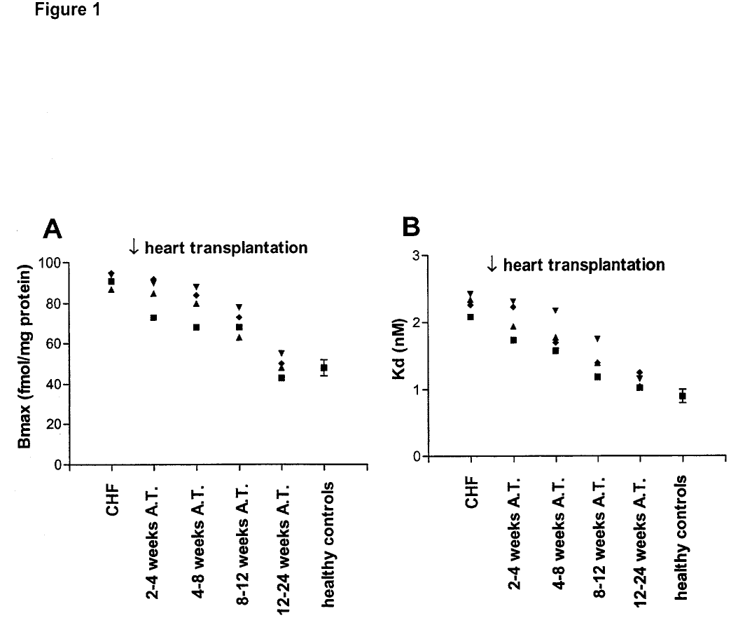

The results show a sensible increase in the number of A_{2A} receptors both in the lymphocytes and neutrophils of patients with heart failure, as demonstrated by the statistically significant increase in Bmax (table 1). This result is in line with the data obtained by the semi-quantitative RT-PCR assessment of A_{2A} receptor expression in the cells circulating in the blood of the same patients. A considerable increase has been observed in the mRNA encoding for the A_{2A} receptor protein (FIG. 1). Binding studies show that the increase in Bmax is also accompanied by a reduction in A_{2A} receptor affinity, as proved by the increase in K_{D} values (table 1). Probably, this variation is a secondary consequence of the considerable increase in the number of receptors. The increase in A_{2A} receptors is also associated with the potentiation of functional coupling of this receptor to its transduction system, as proved by the statistically significant reduction in the EC₅₀ values for NECA in the lymphocytes and neutrophils of patients with heart failure with respect to control subjects (table 2). In the present study, some of the patients with heart failure underwent heart transplantation; the status of the A_{2A} receptor has been determined in the explanted heart using the binding technique. The results show that an increase in Bmax values occurs in the heart tissue, which is analogous to the increase determined in the cells circulating in the blood of the same patients before transplantation (table 3). On the basis of the relevance of this receptor in the modulation of the release of pro-inflammatory cytokines (see: Introduction), which are important for heart damage progression, we believe that the increase in A_{2A} receptor is an attempt to limit the production and release of such cardiotoxic factors.

Preliminary data obtained by the applicants confirm that a progressive normalization of Bmax occurs in the circulating cells of these patients during the months following transplantation, as shown in the Table 4 below: this table shows the binding parameters of [³H]-ZM 241385 radioligand to A_{2A} receptors in lymphocytes and neutrophils membranes taken from control subjects and patients with terminal heart failure (CHF).

TABLE 4

| | Lymphocyte membranes | | Neutrophil membranes | |
|---|---|---|---|---|
| | | B_{MAX} | | B_{MAX} |
| | K_{D} | (fmol/mg | K_{D} | (fmol/mg |
| Subjects | (nM) | protein) | (nM) | protein) |
| 1^{st} CHF | 2.20 | 95 | 2.65 | 85 |
| patient before | | | | |
| transplantation | | | | |
| 2-4 weeks after | 1.70 | 72 | 2.30 | 74 |
| transplantation | | | | |
| 4-8 weeks after | 1.60 | 68 | 1.40 | 70 |
| transplantation | | | | |
| 8-12 weeks after | 1.20 | 62 | 1.32 | 65 |
| transplantation | | | | |
| 12-24 weeks after | 1.05 | 43 | 1.12 | 55 |
| transplantation | | | | |
| 2^{nd} CHF | 2.54 | 88 | 2.26 | 95 |
| patient before | | | | |
| transplantation | | | | |
| 2-4 weeks after | 1.94 | 85 | 2.28 | 85 |
| transplantation | | | | |
| 4-8 weeks after | 1.78 | 80 | 1.42 | 72 |
| transplantation | | | | |
| 8-12 weeks after | 1.40 | 63 | 1.35 | 69 |
| transplantation | | | | |
| 12-24 weeks after | 1.05 | 44 | 1.12 | 51 |
| transplantation | | | | |
| 3^{rd} CHF | 2.43 | 81 | 2.52 | 86 |
| patient before | | | | |
| transplantation | | | | |
| 2-4 weeks after | 2.68 | 96 | 2.45 | 86 |
| transplantation | | | | |
| 4-8 weeks after | 2.18 | 88 | 1.80 | 84 |
| transplantation | | | | |
| 8-12 weeks after | 1.75 | 78 | 1.70 | 73 |
| transplantation | | | | |
| 12-24 weeks after | 1.16 | 55 | 1.22 | 52 |
| transplantation | | | | |
| 4^{th} CHF | 2.28 | 71 | 1.77 | 88 |
| patient before | | | | |
| transplantation | | | | |
| 2-4 weeks after | 2.24 | 92 | 2.18 | 93 |
| transplantation | | | | |
| 4-8 weeks after | 1.70 | 84 | 1.83 | 92 |
| transplantation | | | | |
| 8-12 weeks after | 1.40 | 73 | 1.22 | 67 |
| transplantation | | | | |
| 12-24 weeks after | 1.25 | 50 | 1.16 | 57 |
| transplantation | | | | |

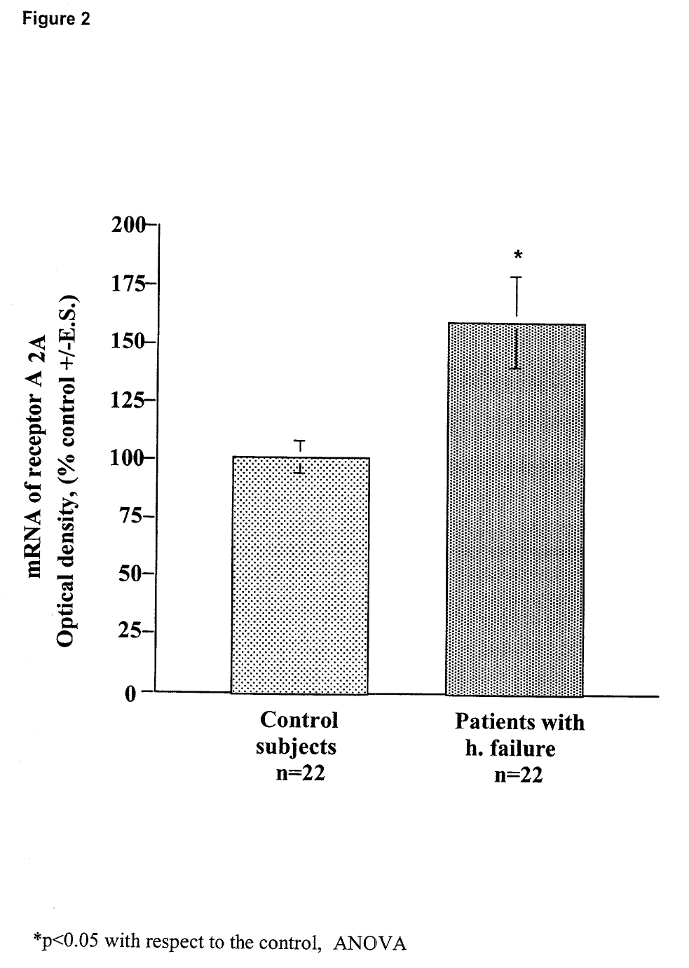

This fact suggests that the normalization of haemodynamic parameters obtained in the patients subjected to heart transplantation also makes this receptor parameter return to normal (FIG. 2).

Globally, these results suggest that (i) the status of the A_{2A} receptor in the circulating cells of the blood reflects the status of this receptor at myocardial level; (ii) the number and expression of this receptor increase in case of heart disease; (iii) this receptor alteration progressively disappears in the transplanted patients. Therefore, the assessment of the A_{2A} receptor at peripheral level provides information both on the status of this receptor in the heart and the extent of the myocardial damage, either in case of heart failure or other types of heart disease of inflammatory nature, such as the myocardial damage associated with transplantation rejection.

### Analysis of the Data and Statistics

The results on binding studies have been analysed using the LIGAND program (Munson P. J. et al., Anal Biochem., 107, 220-239, 1980), which calculates the dissociation constant (K_{D}) and the receptor density (Bmax). The EC₅₀ values of cAMP test have been calculated using the Prism program (Graph Pad, San Diego, Calif.). The data analysis has been carried out using ANOVA, while the differences between controls and CHF subjects have been carried out by means of t of Student for non-matching data. All values were deemed significantly different at P<0.01. Values expressed as mean±SEM.

1. A method to diagnose a heart disease involving haemodynamic deficit, such method comprising the step of measuring, in the patient's affected cells, the density of A_{2A} receptors and/or the production of cyclic AMP induced by a A_{2A} agonist compound.

2. A method according to claim 1, which is used to monitor the onset, and/or development and/or regression of heart disease, the latter occuring upon heart transplantation and/or specific therapeutic protocols.

3. A method according to claim 1, which is used to monitor the efficacy of therapeutic interventions aimed at restoring normal haemodynamic functions.

4. A method according to claim 1, wherein the disease is in its earliest stage of development, in proximity of its complete remission, or in any other situation where its symptoms are hardly visible.

5. A method according to claim 1, wherein the disease is one among terminal heart failure, myocardial infarction, and cardiac hypofunctionality.

6. A method according to claim 5, wherein hypofunctionality occurs after heart transplantation.

7. A method according to claim 1, wherein the affected cells are cells of the heart tissue.

8. A method according to claim 1, wherein the affected cells are cells of the blood.

9. A method according to claim 8, wherein the affected cells are chosen from lymphocytes and/or neutrophils.

10. A method according to claim 1, being performed as follows:
(i) a sample of affected cells is obtained from the patient;
(ii) a part of this sample is tested to assess density of A_{2A} receptors;
(iii) a part of this sample is incubated with an A_{2A} agonist compound and is tested to assess the amount of cyclic AMP produced by these cells;
wherein one between steps (ii) and (iii) is optional and, when both steps (ii) and (iii) are performed, they can take place in any order or simultaneously.

11. A method according to claim 1, wherein the A_{2A} agonist compound is N-ethylcarboxamidoadenoside (NECA) or any other adenosine analogue able to activate A_{2A} receptors.

12. A methods according to claim 1, wherein the production of cyclic AMP is assayed with agents acting at post-receptor level, e.s., at G-protein and/or at adenylyl cyclase level.

13. A method according to claim 1 wherein, during a determined period of time, a series of measurements is taken of A_{2A} receptor density and/or cyclic AMP production, and the respective results are plotted to form a time curve showing the progression of the disease during this period of time.

14. A diagnostic kit for performing the method of claim 1, comprising means for collecting a cells′ sample, means for measuring the density of A_{2A} receptors and/or the production of cyclic AMP, and instruction about the performance of the method of claim 1.
